(19) United States
(12) Patent Application Publication
(10) Pub. No.: US 2022/0340860 A1
(43) Pub. Date: Oct. 27, 2022

(54) AUTOMATED APPARATUS FOR PERFUSION AND REPRODUCIBLE MULTI ORGAN DECELLULARIZATION

(71) Applicant: Atroo, Inc., Newton, MA (US)

(72) Inventors: Abdol-Mohammad KAJBAFZADEH, Tehran (IR); Aram AKBARZADEH, Cambridge, MA (US)

(73) Assignee: Atroo, Inc., Newton, MA (US)

(21) Appl. No.: 17/556,842

(22) Filed: Dec. 20, 2021

Related U.S. Application Data
(60) Provisional application No. 63/127,104, filed on Dec. 17, 2020.

(51) Int. Cl. C12M 1/36; C12M 1/00; C12M 3/00; C12M 1/12; C12M 1/34

(52) U.S. Cl. CPC C12M 41/48; C12M 29/10; C12M 23/48; C12M 31/10; C12M 37/00; C12M 41/14; C12M 41/40

(57) ABSTRACT

A computer-controlled system designed for multi organ decellularization uses continuous organ perfusion for fast, efficient and reproducible organ scaffold preparation under sterile conditions, allowing organ storage for ready organ regeneration. A single organ version is designed to be used for a single organ recellularization using cell, stem cell and autologous cell of organ receiver solutions for minimal or no immunogenic response, mimicking endogenous organ preparation for patients needing transplantation.

### CROSS REFERENCE TO RELATED APPLICATION

This application claims the benefit of U.S. Provisional Patent Application No. 63/127,104, filed Dec. 17, 2020, the contents of which are incorpoprated herein by reference in their entirety.

### FIELD OF THE INVENTION

The present invention relates to a computer-controlled system for multi organ decellularization. More particularly, the invention relates to an automated, programmable system for continuous organ perfusion for fast, efficient and reproducible organ scaffolds preparation under sterile conditions, allowing organ storage for ready organ regeneration.

### BACKGROUND OF THE INVENTION

Many reports describe the tissue engineering decellularization process and its use for tissue transplantation. In principle, tissue and organ decellularization removes all cellular material, antigens, DNA while preserving the three-dimensional architecture of the extracellular matrix (ECM). The organ decellularized ECM scaffold is used to re-cellularize tissues of the body, such as the bladder, artery, esophagus, skin, liver, kidney, trachea, and other organs and tissues.

FIGS. 1A-1C set forth in graphic form the chronological history of tissue engineering.

Generally, the process of decellularization and recellularization has three phases: (1) decellularization: by using ionic and anionic detergents with different timings and concentrations all the native cells are detached from the extracellular matrix (ECM) framework; (2) analyzing the scaffold in order to check the effective preservation of the original texture, to quantify the growth factors present, and to study the scaffold's biological properties; and (3) recellularization: seeding of the scaffold with organ-specific cells, in which cells come directly from the patient who will receive the bioengineered organ (autologous cells), avoiding immunological problems.

For example, U.S. Pat. No. 6,376,244 describes methods for producing a decellularized organ or part of an organ. An isolated organ is mechanically agitated to remove cellular membranes surrounding the isolated organ without destroying the interstitial structure of the organ, and the isolated organ is exposed to a solubilizing fluid that extracts cellular material without dissolving the interstitial structure of the organ and then to a washing fluid that removes the solubilized components, leaving behind a decellularized organ.

In addition, U.S. Pat. No. 8,470,520 describes methods and materials to decellularize an organ or tissue, as well as methods and materials to recellularize an organ or tissue that has been decellularized.

Furthermore, US Patent Application Publication No. 20130109088 describes methods and materials to decellularize an organ or tissue, as well as methods and materials to recellularize an organ or tissue that has been decellularized.

Many other prior art systems accomplish decellularization, as set forth in the literature listed at the end of the specification, but with limited optimization.

For example, in 2012, Sullivan and Mirmalek-Sani developed technology for whole organ decellularization by removing native cellular material from natural vascularized organs mechanically and chemically by perfusing detergents and washing buffers through an intact artery. Concentrated detergent and wash buffer solutions are added to a respective tank and diluted by in line deionized water to appropriate dilutions, and pumped by a peristaltic pump, which can handle up to eight independent perfusion lines per unit. A scaffold's perfusate is routed through interchangeable connectors and a 3-way valve for easy delivery to the scaffolds stored in individual containers within a general solution collecting bin. Solutions enter the renal artery and exit the vein and ureter to either overflow into the general collecting bin and exit via an attached drain or are recirculated within an individual container.

While automation of the decellularization process has been attempted by several research groups, but the automation relates only to semi-automatic control of some bioreactors and perfusion pumps. Thus, although, the word “automated” is used for many of the described systems, these systems are far from having fully-automated and remote computer-controlled operation and/or manipulation. Instead, these so-called automations require constant personnel supervision and suffer from limited performance, synchronization and reproducibility.

Thus, most of reported systems are deficient in their performance to be described as fully automated, Remote-controlled decellurization systems.

It is desirable to provide a computer-controlled system that provides robust automated decellularization to overcome these limitations.

### SUMMARY OF THE INVENTION

In accordance with these and other objects, an automated, programmable system for continuous organ perfusion for fast, efficient and reproducible organ scaffolds preparation under sterile conditions, allowing organ storage for ready organ regeneration is described herein.

Prior to 1996, decellularization used agitation techniques with highly toxic materials, such as DNase, Deoxycolate, RNase, Trypsin, etc., for cell removal from the tissue and organs. These toxic materials were not completely removable from the scaffolds and caused inhibited new cell adhesion and differentiation for tissue recellularization. The inventors hereof previously initiated the development of an automated machine for mass production of scaffolds from different tissues of various species. The primary systems were semiautomatic, manually assisted with temperature-controlled shakers for tissue decellularization of bladder, skin, artery veins, esophagus, trachea and more for pre-clinical research. These preliminary designs, however, did not use perfusion pumps and were inefficient, and adding a perfusion pump improved the decellularization apparatus and used organs such as those the size of rat, rabbit and human organs. However, this earlier apparatus did not completely resolve many pitfalls and suffered from several flaws;

One problem was that decellularizing detergent solutions crystalized following constant perfusion, due to temperature changes that destabilized the decellularization cabin or bioreactor. This crystallization of detergent initiated occlusion of the microvascular tree of the organ and damaged the scaffold structure.

Another problem was that the simultaneous action of the incubator's shaker, peristaltic pumps and refrigeration caused malfunction of various system parts. Therefore, the treatment required a professional operator's supervision during the process.

Furthermore, the temperature variations caused extra cellular matrix damage/integrity and had to be controlled.

The inventors hereof made several modification to this system, including:

1. the number of isolated perfusion bioreactors was increased, and the use of separate perfusion bioreactors added a new dimension for using different organs, as it isolated the separate treatment by the different decellurazing solutions;

2. temperature control for separate bioreactors from +4° C. up to 37° C. was added;

3. lighting systems for proper visualization of organs and fluid levels were added;

4. two separate external pumps were added, a first for perfusion, and a second for fluid drainage for closed system reperfusion;

5. an external waste drainage was added to a removal container at the end of procedure;

6. a fluid reservoir tank was added to the upper cabin to provide closed circulation system that prevent environmental hazards from the toxic detergents;

7. a controlled shaker was added for simultaneous shaking and perfusion; and

8. a pulsatile pump was added to connect up to 14 parallel inlets and outlets valves (each set provided a closed system) allowing a closed system for up to 14 organ perfusion/drainage.

The operation protocol provides for sterilization and quality assurance (QA) of the extra cellular matrix (ECM), a required step prior to cell seeding for transplant of experiment applications. However, in this improved system, the cabins' wash and detergent exchange was still done manually.

The inventors hereof have further improved upon these systems and have developed a fully-automated, fully-programmable, and remotely-controlled system that can perform separate but concurrent decelluraization for different organs.

### BRIEF DESCRIPTION OF THE DRAWINGS

In order for the present invention to be better understood and for its practical applications to be appreciated, the following figures are provided and referenced hereafter. It should be noted that the Figures are given as examples only and in no way limit the scope of the invention. Like components are denoted by like reference numerals.

FIGS. 1A-1C set forth in graphic form the chronological history of tissue engineering;

FIG. 2 is schematically illustrates a front view of a computer-controlled system for multi organ decellularization;

FIGS. 3-5 are perspective, side and back views of the computer-controlled system for multi organ decellularization shown in FIG. 1;

FIG. 6 is a schematic illustration of the fluid pathways within the computer-controlled system for multi organ decellularization shown in FIG. 1; and

FIGS. 7A-7D show a real-life embodiment of the system.

It should be noted that the embodiments depicted are shown only schematically, and that not all features may be shown in full detail or in proper proportion. Certain features or structures may be exaggerated relative to others for clarity. It should be noted further that the embodiments shown are examples only and should not be construed as limiting the scope of the present disclosure or appended claims.

### DETAILED DESCRIPTION OF THE INVENTION

In the following detailed description, various embodiments of the present invention will be described with reference to the accompanying drawings, and numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

In the following description, the same elements will be designated by the same reference numerals although they may be shown in different drawings. Further, various specific definitions found in the following description are provided only to help general understanding of the present invention, and it is apparent to those skilled in the art that the present invention can be implemented without such definitions. Further, in the following description of the present invention, a description of known functions and configurations incorporated herein will be omitted when it may make the subject matter of the present invention rather unclear.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Unless otherwise indicated, the conjunction “or” as used herein is to be understood as inclusive (any or all of the stated options).

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

A smart, synchronized fully computer-controlled, perfusion-bioreactor, serializable single and/or multi organ decellularization system is disclosed herein. As discussed, this system enables full automation and remote computer-control operation and/or programble manipulations, as opposed to prior art systems.

The system provides, robust and reproducible full-organ scaffolds, ready for recellularization. This system improves the reproduction of total organ efficient and cost-effective transplantation.

The disclosed apparatus enables decellularization of small and large organs, accelerating bench-to-bedside medical transplantation and establishes increase the utility of tissue engineering for organ transplantation from live or cadaver donors to the patient in need.

The system allows for programing decellurization of different organs. Organs like the heart, kidney, liver or other tissue require different perfusion flow, detergents, temperature control, time control, organ visualization control, organ wash and system washout post decellurizaion and analytical testing. In addition, fat and other connective tissues may require special attention to avoid complications. The system can be programmed to perform concurrent decellurization of different organs, and the contolled takes into account the particularities required for each organ in setting up the decellurization protocol. This separate remote control system allows separate control of each decellurization process from distance. Camera controls report on the tissue status and provide visual observations throughout the process.

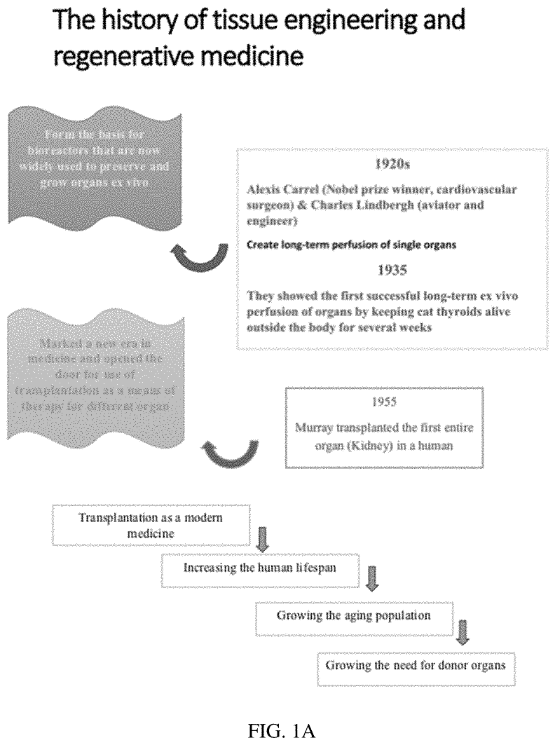

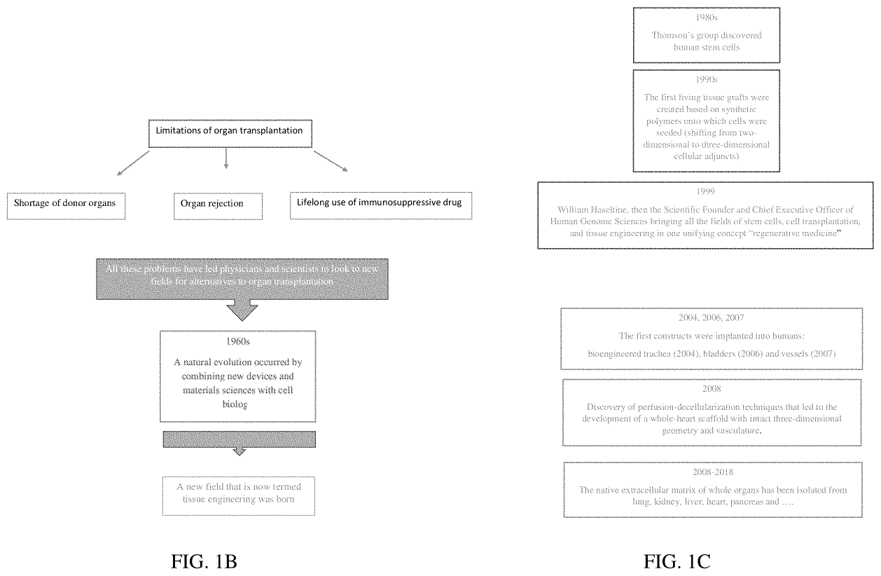

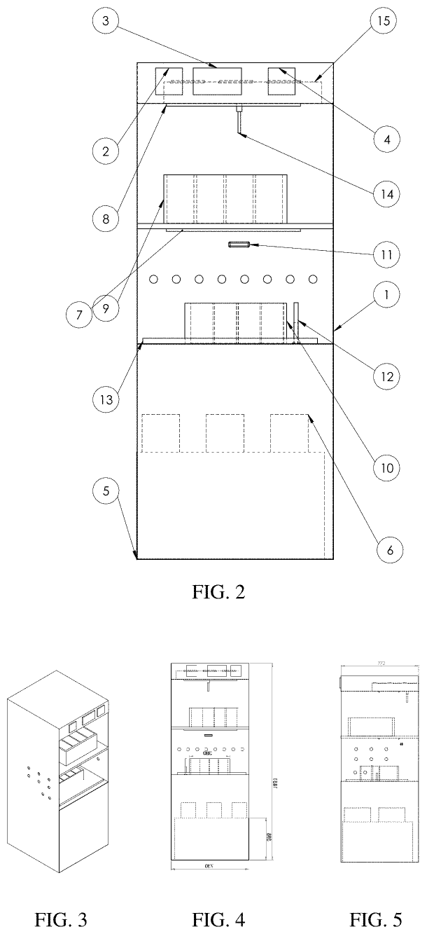

As shown in FIG. 2, the decellularization system includes an incubator 1 with several compartments and levels and with several components contained therein. The decellularization system includes, e.g., on one level thereof, as shown in FIG. 1, a programmer 2, a shaker controller 3 and a temperature controller 4, as well as a control unit 15.

Temperature controller 4 enables the system to have a separate temperature/pressure bioreactor control, from outside the apparatus as well as for remote-control. In one embodiment, temperature controller 4 may be connected to control unit 15 so as to activate mechanical alarms for the thermostat upper and lower limits. In one embodiment, where the enclosure may have a temperature setting of 4 to 20° C., with a precision of 0.1° Celsius.

Shaker controller 3 may be under control of control unit 15, such as remotely or under advance programming, with adjustable speed and operating time.

The apparatus also includes, for example in another compartment, a tank, such as an upper tank 9, under direct illumination by a light 8, e.g., one or more Ultraviolet lights, and under direct monitoring by at least one temperature sensor 14.

The apparatus further includes, for example in another compartment, one or more organ tanks 10, which rest or are positioned on a shaking table 13. The one or more organ tanks 10 can be the sites of separate and concurrent decellurization of one or more organs.

Organ tanks 10 are also monitored by level meter 12, as well as one or more video or static cameras 11, which may require illumination, such as by one or more LED light 7. Each of one or more organ tanks 10 may have its own separate level meter 12, one or more video or static cameras 11, and one or more LED light 7. The system includes a separate photo/video chamber monitoring system, in order to monitor and acquire data during the decellularization process, using an online video and photographs captured from each decellularization phase and production batch protocol. The online monitoring system reduces the human supervision required by present systems.

The apparatus further includes, for example in one or more other compartments, a cooling unit 5, and one or more peristaltic pumps 6. In certain embodiments, the apparatus may contain any number of peristaltic pumps 6, such as six, or fewer or more than six, in order to perform separate and concurrent perfusions, as necessary.

In one embodiment, the system has six separate perfusion pulsatile pumps with multiple electronic programable valves. The proposed system improves on the previously parallel perfusion decellularization system. The perfusion volume and speed as well as duration are entirely similar for each organ by six different perfusion pumps that avoid the variability in infusion/perfusion pressures.

The disclosed apparatus has a programmable, flexible decellularization user-friendly protocol, with advanced remotely-monitored from distance by a smart phone or PC. Following protocol initiation and system set-up, the system machine automatically decellularizes organs in accordance with protocol.

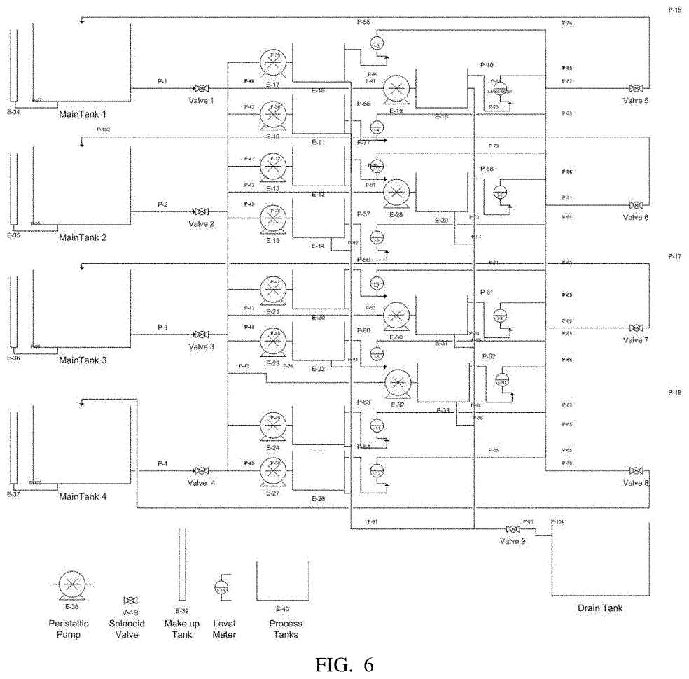

The fully programable computer controlled closed system for single and multi-organ decellularization further includes, as shown in FIG. 6, multiple perfusion control pumps with a recycling decellularization chemicals with draining biohazard storage system. The system also includes multiple recycling valves for intake and outflow liquid perfusion.

The system also has multiple bioreactors for total organ and tissue containers with separately controlled perfusion pumps.

The system provides automated cleaning and washing prior to preparation as well as post decellularization.

The system can be under complete remote-control application, via a smartphone or PC. Data concerning the program protocol performance is recorded.

The system can be programmed for specific organ decellularization, on site or remotely. Similarly, the system can be programmed to decellularize a specific organ, such as the heart, liver, kidney, etc., either for tissue regeneration, for transplantation or for research.

The system encompasses a self-contained, closed system to prevent contamination, including an Ultraviolet light/air sterile system control. The system includes sterile recycled perfusion solutions and a storage for biohazard drainage. In addition, the apparatus could be cleaned automatically and be made ready for the next decellularized organ scaffold preparation.

The system also includes an error alarm notification system. The system sends electronic notification when an error in production is monitored, and the machine will automatically self-abort or stop the procedure until the error is corrected.

FIG. 6 is a schematic illustration of the fluid pathways within the computer-controlled system for multi organ decellularization.

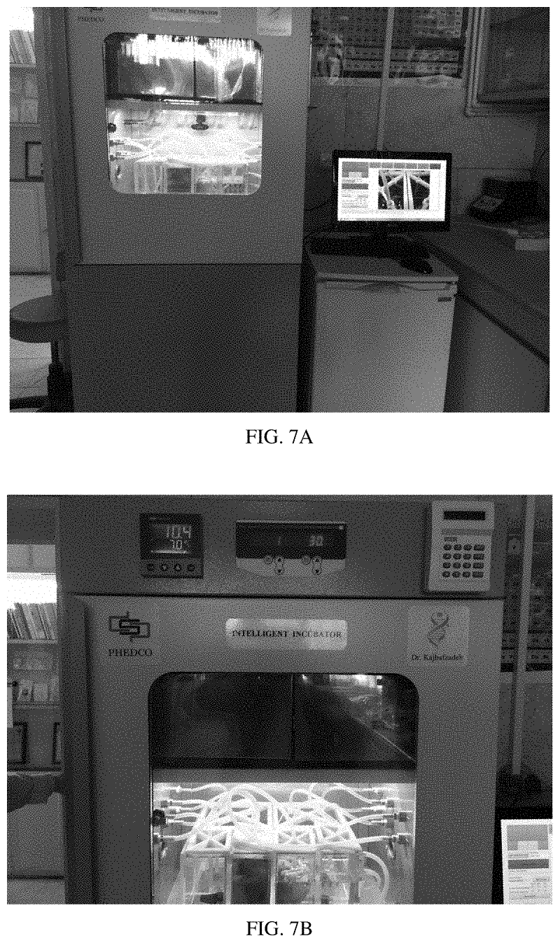

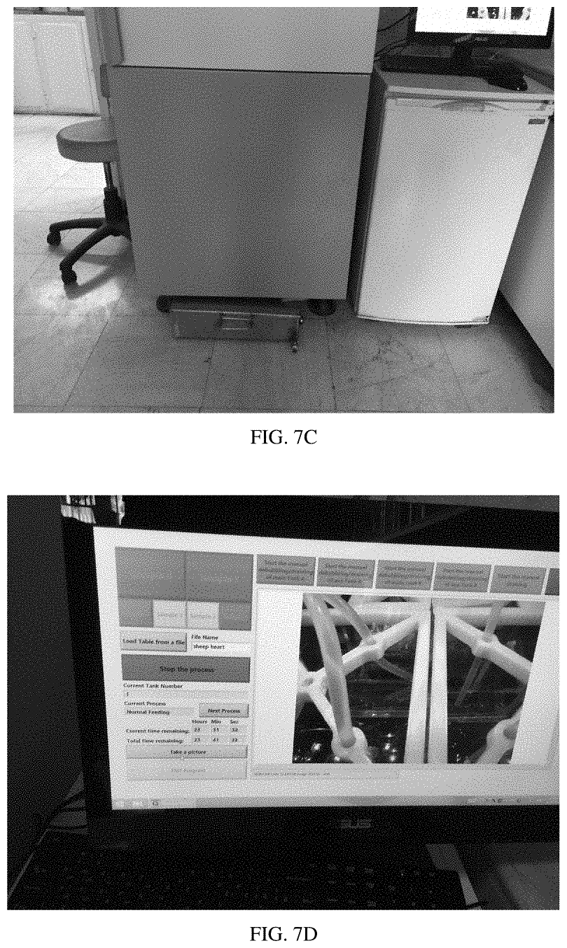

FIGS. 7A-7D show a real-life embodiment of the system. In this embodiment, the capacity of the decellularization apparatus is approximately 125 liters, with inside dimensions of the compartment of 60 cm width×50 cm depth×65 cm height. However, the system can be larger or smaller, as necessary.

In order to implement the method according to some embodiments of the present invention, control unit 15, which may be a computer processor, may receive instructions and data from a read-only memory or a random-access memory or both, including from programmer 2. At least one of aforementioned steps is performed by at least one processor associated with a computer. The essential elements of a computer are a processor for executing instructions and one or more memories for storing instructions and data. Generally, a computer will also include, or be operatively coupled to communicate with, one or more mass storage devices for storing data files. Storage modules suitable for tangibly embodying computer program instructions and data include all forms of non-volatile memory, including by way of example semiconductor memory devices, such as EPROM, EEPROM, and flash memory devices and magneto-optic storage devices.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in base band or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

In some embodiments of the organ and tissue decellularization apparatus, control unit 15 may include an article such as a computer or processor readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein.

Program code embodied on a computer readable medium may be transmitted using any appropriate medium, including but not limited to wireless, wire-line, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++, Python or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram portion or portions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram portion or portions.

The computer program instructions may also be loaded onto a computer, other programmable data processing apparatus, or other devices to cause a series of operational steps to be performed on the computer, other programmable apparatus or other devices to produce a computer implemented process, such that the instructions, which execute on the computer or other programmable apparatus, provide processes for implementing the functions/acts specified in the flowchart and/or portion diagram portion or portions.

It should be understood, with respect to any flowchart referenced herein, that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Different embodiments are disclosed herein. Features of certain embodiments may be combined with features of other embodiments; thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### REFERENCES

1. Baptista P M, Siddiqui M M, Lozier G, Rodriguez S R, Atala A, Soker S. The use of whole organ decellularization for the generation of a vascularized liver organoid. Hepatology 2011; 53(2):604-617.
2. Charest J M, Okamoto T, Kitano K, Yasuda A, Gilpin S E, Mathisen D J, Ott H C. Design and validation of a clinical-scale bioreactor for long-term isolated lung culture. Biomaterials 2015; 52:79-87.
3. Fernandez-Moure J S, Van Eps J L, Rhudy J R, Cabrera F J, Acharya G S, Tasciotti E, Sakamoto J, Nichols J E. Porcine acellular lung matrix for wound healing and abdominal wall reconstruction: A pilot study. Journal of tissue engineering 2016; 7:2041731415626018.
4. Gilpin S E, Guyette J P, Gonzalez G, Ren X, Asara J M, Mathisen D J, Vacanti J P, Ott H C. Perfusion decellularization of human and porcine lungs: bringing the matrix to clinical scale. The Journal of Heart and Lung Transplantation 2014; 33(3):298-308.
5. Gilpin S E, Ott H C. Using nature's platform to engineer bio-artificial lungs. Annals of the American Thoracic Society 2015; 12(Supplement 1):S45-S49.
6. Madariaga M L L, Ott H C. Bioengineering kidneys for transplantation. 2014. Elsevier. p 384-393.
7. Mazza G, Rombouts K, Hall A R, Urbani L, Luong T V, Al-Akkad W, Longato L, Brown D, Maghsoudlou P, Dhillon A P. Decellularized human liver as a natural 3D-scaffold for liver bioengineering and transplantation. Scientific reports 2015; 5:13079.
8. Nichols J E, Niles J, Riddle M, Vargas G, Schilagard T, Ma L, Edward K, La Francesca S, Sakamoto J, Vega S. Production and assessment of decellularized pig and human lung scaffolds. Tissue Engineering Part A 2013; 19(17-18):2045-2062.
9. Olson J L, Atala A, Yoo J J. Tissue engineering: current strategies and future directions. Chonnam medical journal 2011; 47(1):1-13.
10. Ott H C, Clippinger B, Conrad C, Schuetz C, Pomerantseva I, Ikonomou L, Kotton D, Vacanti J P. Regeneration and orthotopic transplantation of a bioartificial lung. Nature medicine 2010; 16(8):927.
11. Ott H C, Matthiesen T S, Goh S-K, Black L D, Kren S M, Netoff T I, Taylor D A. Perfusion-decellularized matrix: using nature's platform to engineer a bioartificial heart. Nature medicine 2008; 14(2):213.
12. Peloso A, Dhal A, Zambon J P, Li P, Orlando G, Atala A, Soker S. Current achievements and future perspectives in whole-organ bioengineering. Stem cell research & therapy 2015; 6(1):107.
13. Pence K A, Mishra D K, Thrall M, Dave B, Kim M P. Breast cancer cells form primary tumors on ex vivo four-dimensional lung model. journal of surgical research 2017; 210:181-187.
14. Price A P, Godin L M, Domek A, Cotter T, D'Cunha J, Taylor D A, Panoskaltsis-Mortari A. Automated decellularization of intact, human-sized lungs for tissue engineering. Tissue Engineering Part C: Methods 2014; 21(1):94-103.
15. Robertson M J, Soibam B, O'Leary J G, Sampaio L C, Taylor D A. Recellularization of rat liver: An in vitro model for assessing human drug metabolism and liver biology. PloS one 2018; 13(1):e0191892.
16. Sanchez P L, Fernandez-Santos M E, Espinosa M A, Gonzalez-Nicolas M A, Acebes J R, Costanza S, Moscoso I, Rodriguez H, Garcia J, Romero J. Data from acellular human heart matrix. Data in brief 2016; 8:211-219.
17. Soto-Gutierrez A, Wertheim J A, Ott H C, Gilbert T W. Perspectives on whole-organ assembly: moving toward transplantation on demand. The Journal of clinical investigation 2012; 122(11):3817-3823.
18. Sullivan D C, Mirmalek-Sani S-H, Deegan D B, Baptista P M, Aboushwareb T, Atala A, Yoo J J. Decellularization methods of porcine kidneys for whole organ engineering using a high-throughput system. Biomaterials 2012; 33(31):7756-7764.
19. Taylor D A, Parikh R B, Sampaio L C. Bioengineering hearts: Simple yet complex. Current stem cell reports 2017; 3(1):35-44.
20. Tsuchiya T, Mendez J, Calle E A, Hatachi G, Doi R, Zhao L, Suematsu T, Nagayasu T, Niklason L E. Ventilation-based decellularization system of the lung. BioResearch open access 2016; 5(1):118-126.

1. A fully programable computer controlled closed system for single and multi-organ decellularization that includes:
a multi prefusion controlled pumps with a recycling decellularization chemicals with draining biohazard storage system;
a multi bioreactors for total organ and tissue containers with separately controlled perfusion pumps;
a multi recycling valves for intake and outflow liquid perfusion;
a separate temperature/pressure bioreactor control;
a UV/Air sterile system control;
an automated cleaning and washing prior to preparation and post decellularization;
a separate photo/video chamber monitoring system;
a data recording of program protocol performance;
a remote control application system (smartphone/PC); and
an error alarm notification system.

2. The system of claim 1 wherein the system is programmable for decellulsarization of specific organs, including heart, liver and kidney, or for just tissue for transplantation or research.
